(19) United States
(12) Patent Application Publication
(10) Pub. No.: US 2007/0043589 A1
(43) Pub. Date: Feb. 22, 2007

(54) Pharmacy benefits design

(75) Inventors: Elizabeth M. Warren, Louisville, KY (US); Timothy B. Sandman, Louisville, KY (US); Jacob M. Hutti, Louisville, KY (US); Jonathan T. Lord, Louisville, KY (US); Julie E. Whitworth, Louisville, KY (US); Elizabeth M. Shaw, Louisville, KY (US); William K. Fleming, Prospect, KY (US); Teresa L. Watson-Heidari, Louisville, KY (US)

Correspondence Address: Markets, Patents & Alliances LLC, 30 Glen Terrace, Stamford, CT, 06906-1401, US

(73) Assignee: Humana Inc.

(21) Appl. No.: 11/119,125

(22) Filed: Apr. 29, 2005

Related U.S. Application Data
(60) Provisional application No. 60/568,517, filed on May 6, 2004.
(60) Provisional application No. 60/572,586, filed on May 19, 2004.
(60) Provisional application No. 60/601,918, filed on Aug. 16, 2004.

(51) Int. Cl. G06F 17/60

(52) U.S. Cl. 705/2

(57) ABSTRACT

A pharmacy benefits plan is designed such that covered scripts are placed into Groups and allowances are provided for the purchase of scripts within each Group. Each Group comprises scripts for pharmaceuticals that provide medical benefits within a given time frame. A first Major Group is assigned to scripts for drugs that are prescribed for conditions that would otherwise result in further medical treatment and expense if said drugs were not taken by an insured. A second Major Group is assigned to scripts for drugs that are prescribed for conditions that would not normally result in subsequent medical costs if the drugs were not taken. Relatively high allowances are provided for scripts in said first Major Group such that the out-of-pocket costs of a given script will not be a barrier to said insured obtaining and taking said scripts. Relatively low allowances are provided for scripts in said second Major Group such that an insured will be further motivated to ask their doctor about low cost alternative drugs for a given treatment.

### CROSS-REFERENCE TO RELATED APPLICATION

This application claims the benefit of the filing date of U.S. provisional patent application Ser. No. 60/568,517, filed May 6, 2004, and entitled “Pharmacy Benefits Design”. Said provisional application is incorporated herein by reference.

This application further claims the benefit of the filing date of U.S. provisional patent application Ser. No. 60/572,586, filed May 19, 2004, and entitled “Pharmacy Benefits Calculator”. Said provisional application is incorporated herein by reference.

This application also further claims the benefit of the filing date of U.S. provisional patent application Ser. No. 60/601,918, filed Aug. 16, 2004, and entitled “Pharmacy Personal Care Account”. Said provisional application is incorporated herein by reference.

Reference Table Submitted on Compact Disk

Table 5 of the present invention is submitted as ASCII file “Rxlmpact Drug List”. A copy of said file is recorded each on compact disks labeled “Copy 1” and “Copy 2” and is incorporated herein by reference. Both compact disks contain the file “Rxlmpact Drug List”. Said copy of said file was created on Apr. 29, 2005 and is 473 kilobytes long.

TABLES FILED ON CD
The patent application contains tables filed on compact disc. These tables have been included at the end of the specification

Table 5 contains 8254 rows and 4 columns. The first row is a header row. Each subsequent row contains data for the cells of that row. A comma (,) separates the data corresponding to the cells of each column in a given row. If a comma is contained between quotes (“), however, then it is part of the text of a cell of the table and does not indicate the separation of the cells of one column from another.

### FIELD OF THE INVENTION

The invention is in the field of health insurance. This invention is more particularly in the field of pharmacy benefits programs.

### BACKGROUND

There is a long felt need for a health insurance plan that can be provided by an employer to their employees such that employees get effective medical treatment for themselves and their families at minimal out-of-pocket costs and employers can keep their costs under control.

A significant factor in the growth of health insurance costs for employers is the high growth in the cost of pharmaceuticals. At first one might expect that the increased use of more effective drugs would lead to a reduction in overall health care costs since more effective drugs should reduce the need for subsequent expensive medical treatments, such as emergency room visits and hospital stays. Experience has not shown this to be the case, however.

Some insurance companies have attempted to control pharmacy costs directly by designing pharmacy benefits plans to encourage insured employees to exercise cost saving behaviors in their pharmaceutical purchases. Some pharmacy benefits plans, for example, provide a low fixed copay for generic drugs and high fixed copay for brand name drugs. The rationale for this is that insured employees will encourage their doctors to prescribe generic drugs as opposed to brand name drugs so that the employees can save on their out-of-pocket costs. Copay pharmacy benefits plans based on a distinction between generic and brand name drugs, however, have done little to stem the growth of pharmaceutical insurance costs. Furthermore, copay plans have encouraged “gaming of the system”. Prescriptions of maintenance drugs for chronic conditions are being offered with unusually large numbers of doses per script so that insured employees that have said chronic conditions can get more of a drug for the same copay. This leads to higher insurance costs for both the employer and the other insured employees that do not have said chronic conditions.

Insurance companies have increased the complexity of pharmacy benefits plans in order to reduce “gaming of the system” but this leads to higher information system costs to administer.

Insurance companies have also designed pharmacy benefits plans with three, four and more Groups of drugs where the insurance benefits provided for a given drug depend upon which of said Groups said drug is assigned to. A drug is assigned to a given Group depending at least in part on the discount that a given insurance company has negotiated with the manufacturer of said drug. Drugs with the largest discounts are assigned to the Groups with the highest benefit levels. The Groups with the highest benefit levels have the lowest out-of-pocket costs for an insured.

Said negotiations of discounts can be costly due to the time and effort involved.

Said negotiations also prevent the assignment of all of the drugs of certain therapeutic classes to the same Groups with the first or second highest benefits levels. Said certain therapeutic classes comprise drugs for high volume maintenance drugs normally prescribed for common chronic conditions. Said chronic conditions would normally lead to high medical costs for a given patient if said drugs were not taken. These certain therapeutic classes include Cholesterol drugs, ACE inhibitors, Heart drugs, Blood pressure control drugs and Proton pump inhibitors. Drug companies require that in order to get their best discount for a given drug, insurance companies must assign their drugs to the Groups with the first or second highest benefits levels. Furthermore, the insurance companies must exclude competing drugs from the same Groups that have the first or second highest benefits levels. Hence there are no pharmacy benefits plans comprising three or more Groups of drugs where all of the drugs offered for a given one of said certain therapeutic classes are in the same Group with the first or second highest benefits levels.

Insurance companies must also provide mechanisms for individual exceptions to a given pharmacy benefits plan so that benefits levels can be adjusted when a given individual's legitimate medical needs are not met by the pharmacy benefits design. A given individual, for example, might legitimately require a brand name drug but cannot afford the high copay. Insurance companies therefore, may provide means for approving and recording individual exceptions. If the exception rate is high, however, these means to provide exceptions can add significantly to the cost of administering a given pharmacy benefits plan.

Hence there is a long felt need in the health insurance industry for a pharmacy benefits design and associated methods and systems that help realize the potential of improved drugs to minimize subsequent medical costs, encourages cost saving prescription drug purchasing behavior by insured employees, minimizes the temptation for gaming of the system, minimizes the need to spend time and effort negotiating discounts for drugs, allows drugs of certain therapeutic classes to all be assigned to the same Group and minimizes the need for individual exceptions.

### SUMMARY OF THE INVENTION

The Summary of the Invention is provided as a guide to understanding the invention. It does not necessarily describe the most generic embodiment of the invention or all species of the invention disclosed herein.

The present invention is a method for providing pharmacy benefits in a health insurance program. The method comprises categorizing pharmacy scripts into at least a first Major Group and a second Major Group. Pharmacy scripts for a given drug are assigned to the first Major Group if they are prescribed for conditions that would otherwise likely require subsequent covered medical costs of an insured person within a given time period if said insured person did not take said drug. Said given time period may be a year.

An antibiotic, for example, might be assigned to said first Major Group. A person might be prescribed an antibiotic if they had an infection. If said person did not take said antibiotic, they might require a subsequent hospital emergency room visit. Said antibiotic might have a cost of $15. Said emergency room visit might have a cost of $600. Hence an employer offering medical insurance to its employees can realize a net overall savings in insurance costs if they could encourage employees to take drugs assigned to said first Major Group.

In order to encourage insured persons take drugs that will result in lower subsequent medical costs in a given time period, the present invention sets benefits payable to an insured person at a first level for scripts assigned to said first Major Group. Said first level of benefits is set so that the retail cost of a drug will not be a barrier to a person obtaining and taking said drug. Hence overall medical costs will be kept lower since said person will be more likely to take the drugs and less likely to require subsequent medical treatment.

Pharmacy scripts that do not result in lower anticipated subsequent medical costs in a given time period are assigned to the second Major Group. Prescription allergy medications, for example, might be assigned to said second Major Group. If a person did not take a prescription for an allergy medication, it is unlikely that they would require subsequent medical treatment in a given time period due to their not taking said drug. Nonetheless, it would be in an employer's bests interest to provide at least some coverage for said drug since it would likely result in an overall increase in employee comfort and productivity if taken.

Benefits payable to an insured person are set at a second level for pharmacy scripts placed in said second Major Group. The second level is set so that there will be some out-of-pocket costs for an insured person taking drugs in said second Major Group. The level of out-of-pocket costs will depend upon which particular drug they are prescribed. Hence an insured person is more likely to encourage their doctor to prescribe the lowest cost effective alternative for a given medical condition so that their out-of-pocket costs are kept low. Hence their insurance carrier and employer's costs will be kept low as well.

The first Major Group may be further divided into Groups covering different time periods between when a drug is prescribed and when subsequent medical expense is likely to occur if said drug is not taken.

The second Major Group may similarly be further divided into Groups. One Group may be for medications that result in improved employee comfort and productivity if taken. Another Group may be for medications that do not result in improved employee comfort and productivity (e.g. hair loss drugs).

### BRIEF DESCRIPTION OF THE DRAWINGS

FIG. 1 is a table of drugs categorized into Group A or “Acute” of the present invention.

FIG. 2 is a table of drugs categorized into Group B or “Chronic” of the present invention.

FIG. 3 is a table of drugs categorized into Group C or “Workplace” of the present invention.

FIG. 4 is a table of drugs categorized into Group D or “Lifestyle” of the present invention.

FIG. 5 is an illustration of the flow and processing of information for a technology enabled method of assigning different Groups to different scripts.

FIG. 6 is an illustration of the flow and processing of information for a technology enabled method of processing a pharmacy claim.

FIG. 7 is an illustration of the flow and processing of information for a technology enabled method of offering a pharmacy benefits plan to a prospective insured.

FIG. 8 is an illustration of the flow and processing of information for a technology enabled method of accumulating and redeeming unused pharmacy benefits.

### DETAILED DESCRIPTION

The following detailed description discloses various embodiments and features of the invention. These embodiments and features are not meant to be limiting.

### Definitions

The definitions provided below are to be applied to their respective terms or phrases as used herein unless the context of a given particular use of a given term or phrase clearly indicates otherwise.

As used herein, the terms “pharmacy script” and “script” refer to a prescription of a given drug or the like.

The terms “benefit”, “benefits” or the like refer to the payment of money to an insured by an insurance company due to a claim made by said insured against a given insurance policy.

The terms “claim”, “claims” or the like refer to a demand made by an insured against an insurance policy for an event that has happened that is covered by said insurance policy.

An “insured” is a person or group of persons covered by an insurance policy.

A “prospective insured” is a person or group of persons to whom an insurance policy is offered.

The phrase “pharmacy benefits plan” or the like refer to the terms of an insurance policy that covers the pharmacy needs of an insured.

The term “Group” refers to a category of pharmacy scripts according to the present invention.

The term “Class” refers to a group of scripts that are prescribed for the same or similar therapeutic effects.

The term “allowance” refers to the amount of money that an insurance company will pay as a benefit for a covered script. If the cost of the script exceeds the allowance, then the insured pays the difference. The difference is an out-of-pocket expense.

The phrase “adjudication of a claim” or the like refers to the process for determining if a claim is covered by an insurance policy, such as a pharmacy benefits plan.

A “pharmacy claim adjudicator” is an entity, such as a company, that performs adjudication of pharmacy claims on behalf of one or more insurance companies.

### Exemplary Embodiment

In one embodiment of the present invention, pharmacy scripts are categorized into one of four Groups. The names of the Groups are “Acute”, “Chronic”, “Workplace”, and “Lifestyle”. The Groups are also referred to as Group A, Group B, Group C and Group D respectively. Groups A and B are subgroups of the first Major Group described above. Groups C and D are subgroups of the second Major Group described above.

In an alternate embodiment of the invention, pharmacy scripts are categorized into one of five Groups. The names of the Groups are “Acute”, “Chronic”, “Workplace”, “Lifestyle”, and “Not Covered”. The Groups are also referred to as Group A, Group B, Group C, Group D and Group 0 respectively.

### Acute Group

Pharmacy scripts are assigned to the Acute Group if they are prescribed for medical conditions that would otherwise likely require subsequent covered health care costs for an insured person within one year if they were not taken.

Antibiotics such as amoxicillin, for example, are categorized as Acute. A doctor normally prescribes antibiotics for a person who suffers from an infection. If the person did not receive the antibiotics, then the infection would likely get worse and the person would likely require subsequent significant medical costs such an emergency room visit or a hospital stay within a year. A prescription of antibiotics might cost $15. An emergency room visit to treat an infection might cost $600 and an overnight stay in a hospital might cost $2,000.

An employer providing medical benefits covering both pharmaceutical costs, emergency room costs and hospital costs will have lower insurance costs in a given year, and hence be able to offer more total benefits at a given premium, if insureds with infections are encouraged to take antibiotics when prescribed. The present invention seeks to accomplish this by offering a relatively high level of pharmacy benefits for antibiotics as well as other scripts in the Acute Group so that the out-of-pocket costs of the scripts is not a barrier to the insured taking the prescribed drugs.

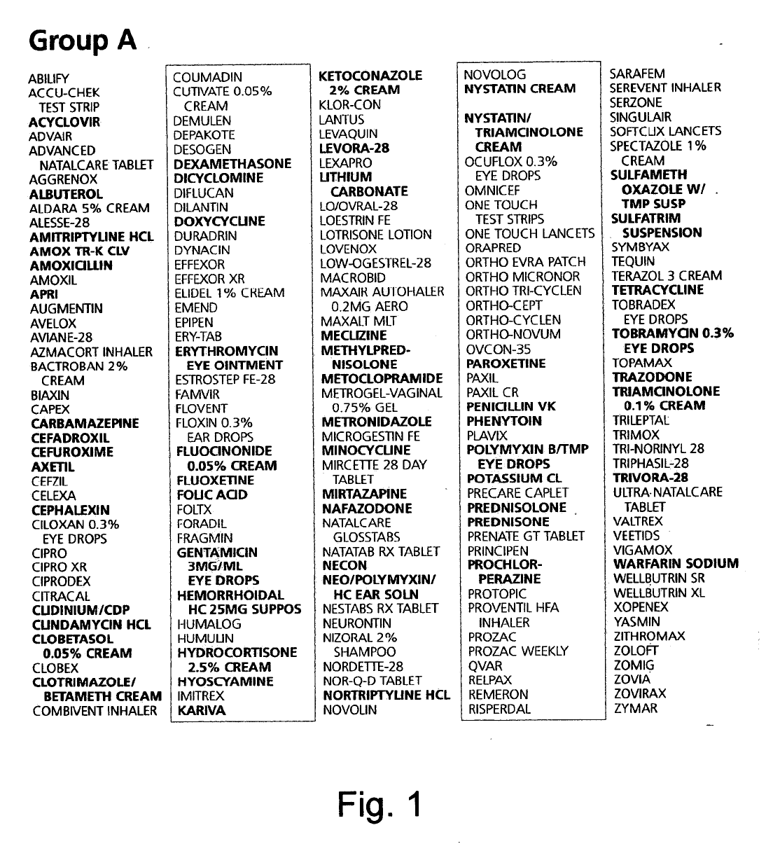

FIG. 1 is a list of drugs that may be categorized as Acute (Group A). This list can be provided to prospective insureds so that they can see if the particular drugs they take fall into Group A. This list does not cover all drugs in Group A, but just the most popular. These drugs account for roughly 80% of the total spend for Group A drugs as experienced by a given population of insured.

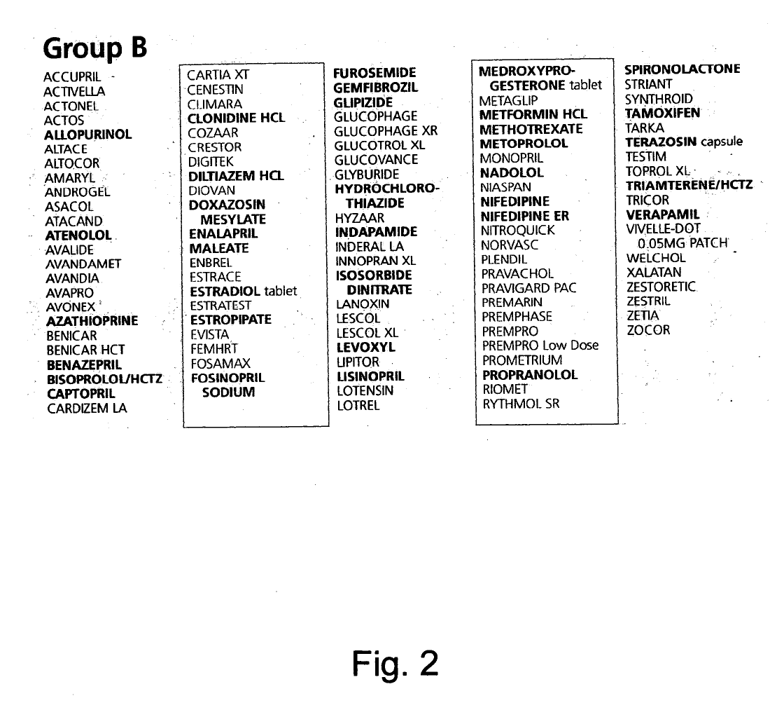

FIGS. 2, 3, and 4 show lists of drugs for Groups B, C and D respectively. Similar to FIG. 1, these lists display the most popular drugs and represent roughly 80% of the spend for a given Group.

The lists of drugs in FIGS. 1 to 4 can be used to generate a list of scripts that are categorized in to Groups. Said list would cover about 80% of the total spend and would be at least partially useful for carrying out the current invention if the scripts for drugs that were not categorized into Groups were placed into a default Group.

The benefits paid for pharmacy scripts categorized as Acute should be high enough to generally cover the cost of most effective alternatives, but not necessarily the most expensive effective alternatives.

The average cost of pharmacy scripts categorized as Acute is about $40 in the United States as of 2001. A suitable benefit level for pharmacy scripts categorized as Acute is an allowance of $30 per script.

A $30 allowance is 75% of the average cost of Acute Scripts for this example. Suitable allowances can be in the range of $20 to $40, or 50% to 100% of the average. Allowance may even be as low as 25% of the average and as high as 150% of the average. An allowance of $30, for example, is sufficient to cover the $15 cost of an effective script of a generic antibiotic. It is not, however, sufficient to fully cover the $45 cost of a name brand antibiotic such as Z Pak®. Z Pak® is a product of Pfizer, Inc. of New York, N.Y. Z Pak® is marketed as being more convenient to take than a generic antibiotic, but it is not necessarily more effective for a given infection. Z Pak® requires one dose per day whereas a generic antibiotic might require 4 doses per day. An insured person who is concerned about effective treatment at their lowest out-of-pocket cost might encourage their doctor to prescribe a generic antibiotic. An insured person who is willing to pay some out-of-pocket costs for a more convenient option than the generic, might encourage their doctor to prescribe Z Pak®. In either case, the out-of-pocket cost of either antibiotic should not be barrier to taking them.

### Chronic Group

Pharmacy scripts for a given drug are placed into the Chronic Group if said drug is prescribed for medical conditions that would require subsequent health care costs in a year or more if the drug were not taken. FIG. 2 is a list of drugs that may be categorized as Chronic (Group B). Cholesterol reduction drugs such as Lipitor, for example, are categorized as Chronic.

A doctor normally prescribes cholesterol reduction drugs for a person who suffers from a blood cholesterol level above a certain threshold. A person with cholesterol levels above said threshold are considered to have a higher than normal probability of heart disease in a period greater than one year, such 5 years or more. If the person did not take the cholesterol reducing drugs, then the health of their heart would deteriorate over a period of years and they would then likely incur significant medical costs, such an emergency room visit or a hospital stay.

An employer offering medical benefits to employees wherein the employees had an anticipated average tenure with the employer of more than a year, could reduce the cost of providing said medical benefits, or could offer a higher level of benefits at the same cost, if coverage were provided for scripts in the Chronic Group such that out-of-pocket cost was not a significant barrier to them being taken.

Benefits paid for pharmacy scripts categorized as Chronic should be high enough to generally cover the cost of some effective alternatives, but not necessarily all alternatives. It is expected that the insured will often have some out-of-pocket expenses for scripts categorized as Chronic.

The average cost of pharmacy scripts categorized as Chronic in the United States as of 2001 is about $40. A suitable benefit level for pharmacy scripts categorized as Chronic is a $20 allowance per script, or 50% of the average. A suitable range of allowances can be 0$ to $30, or 0% to 75% of the average.

Employers that anticipate long tenures of employees might offer allowances at a higher level, such as 100%. Employers that anticipate short tenures of employees might offer lower allowance levels. Employers that anticipate tenures of a year or less might offer an allowance of $0.

For example, an employer with an anticipated average tenure of 10 years for their employees might offer a $30 allowance per script.

An insured employee or their dependent that required a cholesterol reducing drug might choose between the brand name drug, Mevacor® produced by Merck & Co., Inc, of Whitehouse Station, N.J., or the generic equivalent Lovastatin. A one month's supply of Mevacor® costs $63.75. If the allowance for a month's supply of a Chronic drug was $20, then the insured's out-of-pocket expense for Mevacor® would be $43.75 per month. A one month's supply of Lovastatin, however, costs $32.70. The allowance for Lovastatin is the same as for Mevacor®, hence the insured's out-of-pocket expense for Lovastatin would only be $12.70 per month. The low out-of-pocket costs for the effective generic treatment would encourage insureds to take the treatment, even if they had limited financial means. Insureds of higher financial means might choose the name brand treatment if, for example, they had the perception, justified or not, that the name brand treatment was of a higher quality than the generic treatment. An employer might thus anticipate that employees will have sufficient pharmacy benefits such that they would take at least the effective low cost alternative and thus reduce the employer's anticipated subsequent medical costs from their relatively long tenure employees.

The Chronic Group might be further subdivided into Subgroups for drugs that have a medium term benefit in reducing subsequent health costs, such as 1 to 5 years and for drugs that have a longer term benefit in reducing subsequent health costs, such as 5 years or more. Thus employers offering health care plans based on the invention can more closely tune the benefits levels of each Group and Subgroup to meet the anticipated tenure of their employees. Additional Subgroups for Acute can be similarly established.

### Workplace Group

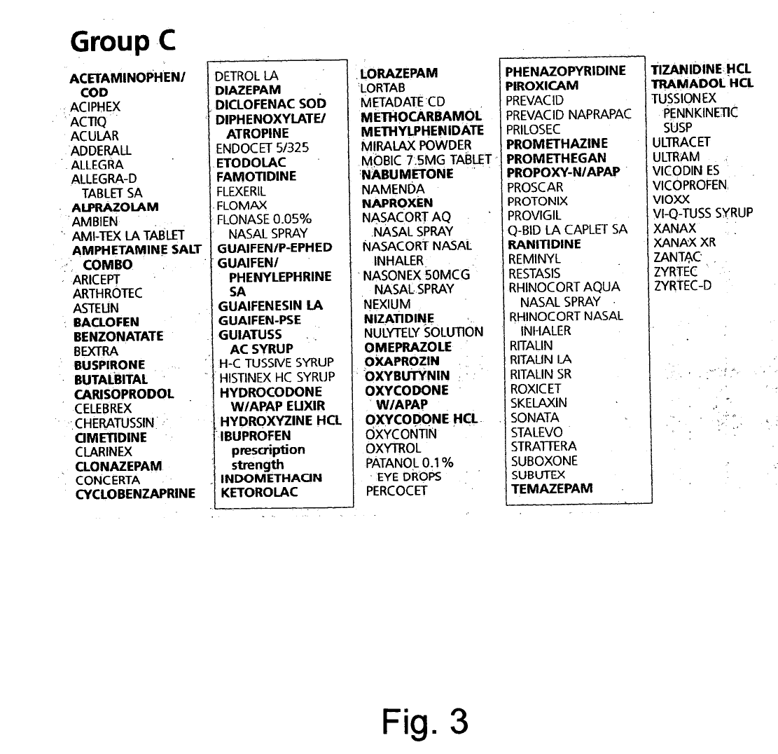

Pharmacy scripts are placed into the Workplace Group if they are prescribed for medical conditions that would otherwise likely reduce a person's productivity on the job, but would not usually result in future health if left untreated. FIG. 3 is a list of drugs that may be categorized as Workplace (Group C). Non-sedating antihistamines such as Allegra, for example, can be placed in the Workplace Group.

A doctor normally prescribes non-sedating antihistamines for a person who suffers from chronic or seasonal allergies. A person suffering from allergies will have lower productivity at work and may have higher absenteeism. An employer providing benefits for non-sedating anti-histamines will get a return on their investment in pharmacy benefits in the form of higher worker productivity or lower absenteeism, but not necessarily lower medical costs. Employers will also earn more goodwill from their employees if a significant benefit is provided for scripts that treat maladies that interfere with their employees' productivity or personal comfort.

Benefits paid for pharmacy scripts categorized as Workplace should be high enough to cover a significant fraction of the cost of the scripts, but low enough so that the insured also pays a significant fraction of the cost.

The average cost of pharmacy scripts categorized as Workplace in the United States as of 2001 is about $40. A suitable benefit level for pharmacy scripts categorized as Workplace is an allowance of $10 per script, or 25% of the average. A suitable range of benefits is a $0 to $30 allowance or 0 to 75% of the average.

An employer might offer a large allowance for scripts in the Workplace Group if a significant fraction of their employees have safety critical jobs. Airlines, for example, might offer an allowance of $30 to their pilots for scripts in the Workplace Group. It would be worthwhile for the airlines to invest in medically necessary non-sedating antihistamines such that they would have a higher assurance of alert pilots who were not distracted by allergies and hence were less likely to have an accident due to the allergies. Similarly, trucking companies might offer a high allowance for scripts in the Workplace Group as an investment in maintaining a better safety record among their drivers.

### Lifestyle Group

Pharmacy scripts are placed into the Lifestyle Group if they are prescribed for a person's personal medical condition, but that condition would not result in lower subsequent covered medical costs or higher worker productivity if left untreated.

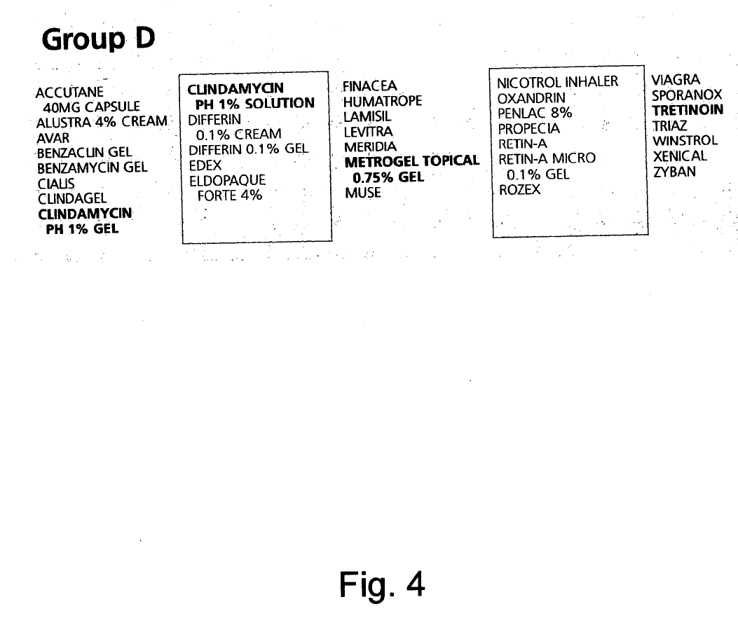

FIG. 4 is a list of drugs that may be categorized as Lifestyle (Group D). Erectile dysfunction drugs such as Cialis, for example, are categorized as Lifestyle.

A doctor normally prescribes erectile dysfunction drugs for a person who suffers from erectile dysfunction. A person with erectile dysfunction will not normally incur subsequent health care costs or suffer from significantly reduced productivity related to their employment if the condition is not treated. Nonetheless, an employer offering some benefit to cover erectile dysfunction drugs will generate good will among their employees. In particular, an employee might not normally expect there to be any coverage for said drug. Hence if some coverage is provided, the employee might consider said coverage to be a valuable perk offered by their employer.

Benefits paid for pharmacy scripts categorized as Lifestyle should be high enough to be viewed as significant by the insured, but low enough such that the insured pays most of the cost.

The average cost of pharmacy scripts categorized as Lifestyle in the United States as of 2001 is about $60. A suitable benefit level for pharmacy scripts categorized as Lifestyle is a $5 allowance per script, or 10% of the average. A suitable range of allowances is 0$ to $20, or 0 to 33% of the average cost per script.

The Lifestyle Group can also be a default Group that pharmaceuticals are assigned to if they are new or if it is not clear which Group they otherwise belong into.

### Not Covered

Pharmacy scripts are placed into the Not Covered Group (Group 0) if they are not covered by a pharmacy benefits plan. General anesthetics, for example, are categorized as Not Covered.

A doctor normally prescribes general anesthetics for patients undergoing surgery. This medical cost is normally already covered by the insured's medical insurance and therefore is not additionally covered by the insured's pharmacy benefits plan.

Over-the-counter drugs are another example of drugs that are not normally covered by an insured's pharmacy benefits plan. The insured normally bears 100 percent of the cost of over-the-counter drugs.

Benefits Plan Design—Allowance

One method for providing benefits to an insured according to the present invention is to provide an allowance for scripts wherein the allowance level is selected based on the Group that a script falls into. Table 1 below describes different allowance levels that are suitable for different scripts given the average cost of scripts in a given Group.

TABLE 1

| | | Range of | |
|---|---|---|---|
| | Allowance per | Suitable | Average cost |
| Group | script | Allowance | of script |
| Acute | $30 | $20-$40 | $40 |
| Chronic | $20 | $0-$30 | $40 |
| Workplace | $10 | $0-$30 | $40 |
| Lifestyle | $5 | $0-$20 | $60 |

### Alternative Benefits—Copay

An alternative method of providing benefits for scripts categorized as Acute, Chronic, Workplace and Lifestyle is for the insured to pay a co-payment (i.e. copay) for each script purchased and for the insurance company to provide a benefit equal to the difference between the price of the drug and the co-payment. There may also be an annual deducible that must be met before the insurance company pays any benefits.

Table 2 below give a schedule of suitable co-payments with and without an annual deductible for scripts that have the given average prices indicated.

TABLE 2

| | Co-payment | Co-payment | |
|---|---|---|---|
| | with no | with $100 | Average cost |
| Group | deductible | deductible | of script |
| Acute | $10 | $5 | $40 |
| Chronic | $25 | $20 | $40 |
| Workplace | $50 | $40 | $40 |
| Lifestyle | 25% of cost of | 25% of cost of | $60 |
| | script | script | |

The benefit levels provided for the instant invention, including those indicated in Tables 1 and 2, will scale according to the average price per script in each Group for the monetary units and other appropriate demographics of the insured population. If the averages are lower in a given location, then the allowances, for example, may be lower.

Various features of the allowance method and the co-payment method of providing benefits can be combined. For example, a benefit method might comprise an allowance for scripts of an Acute Group and a co-payment method for the Chronic Group.

### Method for Assignment of Scripts to Different Groups

In order to apply the method of the present invention, each script of a given type in a pharmacopoeia that is covered by a pharmacy benefits plan should be assigned one of the Groups. A suitable method for assignment is to maintain a lookup table for each script. The lookup table contains an identifier of the script and the script's associated Group. A suitable identifier for each script is its National Drug Code (NDC) as assigned by the United States Food and Drug Administration. A description of the NDC can be found at www.fda.gov/cder/ndc/index.htm (last viewed Apr., 12, 2004). Said web page and the contents of all of the links to said web page are incorporated herein by reference.

An archived description of the NDC can be found at:

http://web.archive.org/web/20030202075941/http://www.fda.gov/cder/ndc/index.h tm Said web page was archived Feb. 2, 2003 and was last viewed on Apr. 6, 2004. Said web page and the contents of all of the links to said web page are incorporated herein by reference.

Said lookup table may be stored in a database on a general-purpose computer. Provision can be made for an entity adjudicating pharmacy claims to access the lookup table to determine benefit levels for individual scripts that are being adjudicated.

The therapeutic effect of each script may be used to determine which Group a script belongs in. Table 3 below gives an example of Classes of therapeutic effects and which Groups the scripts of a given Class belong to. The therapeutic Classes are listed below each Group. The Groups are listed in the first row.

The Classes are well known descriptions of the therapeutic effects of different drugs.

TABLE 3
Therapeutic Classes assigned to different Groups

| | Group A: | Group B: | Group C: | Group D: |
|---|---|---|---|---|
| | Acute | Chronic | Workplace | Lifestyle |
| Therapeutic Class | Acute electrolyte | Androgens | Alzheimer's drugs | Acne drugs |
| | drugs | | | |
| | Allergic reaction | Chemotherapy drugs | Amphetamines—ADHD | Complementary |
| | steroids | | drugs | alternative medicine |
| | | | | drugs |
| | Anaphylaxis drugs | Cholesterol drugs | Analgesics | Cosmetic drug |
| | Antibiotics | Cystic Fibrosis drugs | Anti-anxiety drugs | Growth Hormone |
| | | | | drugs |
| | Anti-convulsants | Estrogens | Anti-diarrheal drugs | Lifestyle drugs; e.g. |
| | | | | Erectile Dysfunction |
| | Anti-depressants | Gout drugs | Cough/Cold/Allergy | Obesity drugs |
| | Antidote drugs | Heart drugs; e.g. blood | Influenza | Smoking cessation |
| | | pressure control drugs | | drugs |
| | Anti-emetics | Irritable bowel | Laxatives | |
| | | syndrome drugs | | |
| | Anti-Microbial-PPI | Metabolic Disease | Muscle relaxation drugs | |
| | combination drugs | drugs; e.g. bone loss, | | |
| | | enzyme need | | |
| | Anti-Psychotics | Multiple sclerosis drugs | Narcotics | |
| | Asthma drugs | Ophthalmic—Glaucoma | Non-sedating | |
| | | drugs | antihistamines | |
| | Blood Clotting | Oral diabetic drugs | Non-Steroid Anti- | |
| | Modifiers | | inflammatory drug | |
| | Contraceptives | Thyroid drugs | Ophthalmic— | |
| | | | Decongestants | |
| | Diabetic | | Parkinson's disease | |
| | supplies/insulin | | drugs | |
| | Hematologic | | Prostate Hypertrophy | |
| | Growth Factors | | | |
| | HIV drugs | | Proton Pump drugs/H-2 | |
| | | | drugs used to reduce | |
| | | | stomach acid. | |
| | Migraine drugs | | Topical Anesthetics | |
| | Ophthalmic— | | | |
| | Steroids and Anti- | | | |
| | infectives | | | |
| | Otic—anti- | | | |
| | infectives | | | |
| | Rx Vitamins/ | | | |
| | prenatal | | | |
| | Topical—anti- | | | |
| | infectives | | | |

A person skilled in the art of pharmaceutical sciences (e.g. one with a Pharm. D. degree from an accredited institution) can assign scripts to the therapeutic Classes according to the known therapeutic effects of the drugs. A suitable default Group for drugs that do not appear to fall into one of the Classes above is Group D: Lifestyle.

Table 5, provided herein on CD ROM, is a list of scripts that have been assigned according to the above method into the five different Groups, Group A, Group B, Group C, Group D and Group 0. Each row of Table 5 indicates the names and Group of a given script. The first column of Table 5 is labeled “Group” and indicates the Group that a given script falls into. Column 2 of Table 5 is labeled “Drug Brand Name” and indicates the brand name of a given script. Column 3 of Table 5 is labeled “Drug Generic Name” and indicates the generic name of a given script. Column 4 of Table 5 is labeled “Drug Label Name” and indicates the label name of a given script.

### Automated Method of Assignment of Groups to Scripts

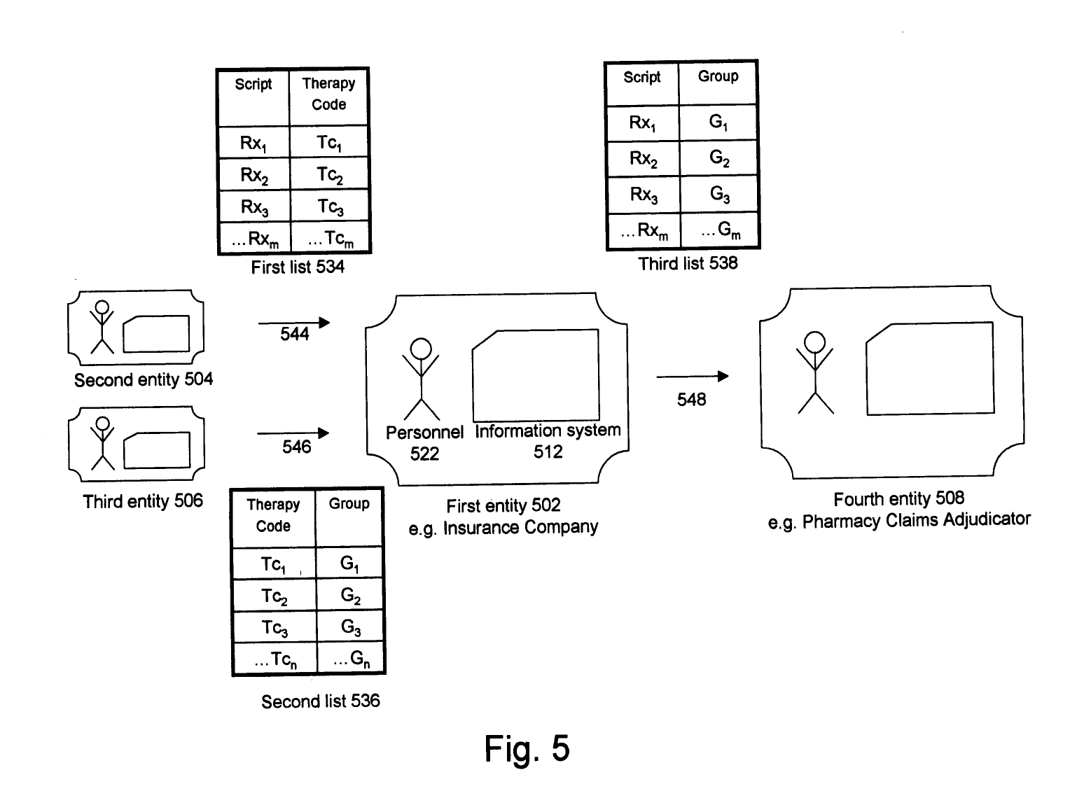

A method for the automated assignment of different Groups to different scripts is illustrated in FIG. 5. The assignment may be performed by an entity 502, such as an insurance company. Said entity does not have to be an insurance company, however. It can be any entity capable of performing the method.

Entities are shown in the FIGS. 5 to 8 as rectangles with recurved corners. An example is entity 502, the insurance company.

Entities may comprise one or more personnel. An example is element 522, the personnel of said insurance company. Said personnel are collectively shown as a stick figure.

Entities may further comprise information systems. An example is element 512, the information system owned by or under the control of said insurance company 502. Information systems are shown as rectangles with a truncated corner.

Information systems may comprise one or more of computers, servers, input devices, output devices, data storage devices, telecommunications equipment and software. Information systems may communicate with other communications systems via telecommunications means, such as the Internet.

Information systems may also communicate with personnel via input/output devices. Persons may communicate with other persons using information systems.

Information transfer is shown as arrows. Information itself is shown as lists.

An example of the step of information transfer is element 544. An example of the information transferred is element 534. Element 544 indicates the transfer of list 534 from entity 504 to entity 502.

The description of lists and entities as separate elements FIGS. 5 to 8 is strictly for the convenience of explanation. Any number of entities or lists may exist as a single element, elements that are subsets of each other or elements that are aggregates of each other. For example, entity 506 may be a department of entity 502. Entity 504, on the other hand may be a vendor to entity 502. Entity 508 may be an aggregate of multiple entities that work together to perform a particular function.

Referring to FIG. 5, a method of assigning pharmacy scripts into Groups, said Groups being useful to a pharmacy claims adjudicator 508 for determining the benefits payable for one or more pharmacy claims made by an insured person, comprises a first entity 502:
a) receiving 544 from a second entity 504 a first list 534 of said scripts, said list comprising therapy classification codes Tcₘ assigned to at least a portion of said scripts Rxₘ;
b) receiving 546 from a third entity 506, a second list 536 of said therapy classification codes, said list comprising Groups Gₙ assigned to at least a portion of said therapy classification codes Tcₙ wherein;
   i. said Groups comprise a first Major Group;
   ii. said therapy classification codes assigned to said first Major Group correspond to scripts for drugs that are prescribed for medical conditions that would otherwise likely require subsequent medical costs within one year of being prescribed if said drugs were not taken by said insured when prescribed; and
   iii. said first Major Group is assigned to at least two or more different therapy classification codes in said second list; and
c) providing 548 a third list 538 to said pharmacy claims adjudicator, said third list being formed by assigning said Groups of said second list to said scripts of said first list using said therapy classification codes as a key;
   wherein at least one of said steps is carried out at least in part by an information system.

Said pharmacy claims adjudicator would use said third list to provide the concrete useful and tangible result of adjudicating pharmacy claims made by an insured.

A preferred type of therapy code suitable for use in lists 534 and 536 is one wherein said therapy codes have a hierarchical structure. The United States Food and Drug Administration, for example, publishes a database called “Drug Class Data” wherein each script in the National Drug Code Directory is assigned to a four-digit drug therapy class (i.e. PRODUCT_CLASS_NO). Said drug therapy class is based on the labeled indication of a given script and indicates the general therapeutic function of the drug. The first two digits of said drug therapy class indicate the major therapy class of a drug. The second two digits indicate the minor therapy class of a drug. Drug therapy classes with “00” in the last two digits encompass an entire major class.

A surprising benefit of using a hierarchical drug therapy classification scheme is that new drugs can be easily assigned to a major class and hence get properly assigned to a Group before they are assigned to a more specific drug therapy class. Hence at any given time, the assignment of scripts to Groups can be more easily kept up to date.

Table 4 below lists the major and minor FDA drug therapy classes and a suitable assignment of these classes to the Groups A, B, C, D and 0 of the present invention. This list may be used as said second list 536 in the above described method for automatically assigning Groups to scripts.

The first column of Table 4 is labeled “PRODUCT_CLASS_NO” and indicates an FDA drug therapy class.

The second column of Table 4 is labeled “FDA drug class” and indicates the FDA drug therapy class common name.

The third column is labeled “Group” and indicates the Group that scripts falling into a given FDA drug therapy class are to be assigned to.

If more than one Group is specified in Table 4 for a given FDA drug therapy class, then any one of said Groups is suitable for that class.

Multiple suitable Groups can arise when some of the scripts of a given FDA drug class would be assigned to one Group, and other scripts in the same FDA drug class would be assigned to a different Group.

As discussed below, the particular Group chosen for a given Class will represent the best compromise for a given embodiment of the invention.

TABLE

| PRODUCT_CLASS_NO | FDA drug class | Group |
|---|---|---|
| 0117 | ANESTHETICS, LOCAL | 0 |
| 0118 | ANESTHETICS, GENERAL | 0 |
| 0119 | ANESTHESIA, ADJUNCTS TO/ANALEPTICS | 0 |
| 0120 | MEDICINAL GASES | 0 |
| 0121 | ANESTHETICS, TOPICAL | C |
| 0122 | ANESTHETICS, OPHTHALMIC | A |
| 0123 | ANESTHETICS, RECTAL | C |
| 0200 | ANTIDOTES | A |
| 0281 | ANTIDOTES, SPECIFIC | A |
| 0283 | ANTIDOTES, GENERAL | A |
| 0285 | ANTITOXINS/ANTIVENINS | A |
| 0286 | ANAPHYLAXIS TREATMENT KIT | A |
| 0300 | ANTIMICROBIALS | A |
| 0346 | PENICILLINS | A |
| 0347 | CEPHALOSPORINS | A |
| 0348 | LINCOSAMIDES/MACROLIDES | A |
| 0349 | POLYMYXINS | A |
| 0350 | TETRACYCLINES | A |
| 0351 | CHLORAMPHENICOL/DERIVATIVES | A |
| 0352 | AMINOGLYCOSIDES | A |
| 0353 | SULFONAMIDES/RELATED COMPOUNDS | A |
| 0354 | ANTISEPTICS, URINARY TRACT | A |
| 0355 | ANTIBACTERIALS, MISCELLANEOUS | A |
| 0356 | ANTIMYCOBACTERIALS (INCL ANTI LEPROSY) | A |
| 0357 | QUINOLONES/DERIVATIVES | A |
| 0358 | ANTIFUNGALS | A |
| 0388 | ANTIVIRALS | A |
| 0400 | HEMATOLOGICS | A |
| 0408 | DEFICIENCY ANEMIAS | 0, A |
| 0409 | ANTICOAGULANTS/THROMBOLYTICS | A |
| 0410 | BLOOD COMPONENTS/SUBSTITUTES | A |
| 0411 | HEMOSTATICS | A |
| 0500 | CARDIOVASCULAR—RENAL | B |
| 0501 | CARDIAC GLYCOSIDES | B |
| 0502 | ANTIARRHYTHMICS | B |
| 0503 | ANTIANGINALS | B |
| 0504 | VASCULAR DISORDERS, CEREBRAL/PERIPHERAL | B |
| 0505 | HYPOTENSION/SHOCK | B |
| 0506 | ANTIHYPERTENSIVES | B |
| 0507 | DIURETICS | B |
| 0508 | CORONARY VASODILATORS | B |
| 0509 | RELAXANTS/STIMULANTS, URINARY TRACT | C |
| 0510 | CALCIUM CHANNEL BLOCKERS | B |
| 0511 | CARBONIC ANHYDRASE INHIBITORS | B |
| 0512 | BETA BLOCKERS | B |
| 0513 | ALPHA AGONISTS/ALPHA BLOCKERS | B |
| 0514 | ACE INHIBITORS | B |
| 0600 | CENTRAL NERVOUS SYSTEM | A, C, D |
| 0626 | SEDATIVES/HYPNOTICS | C |
| 0627 | ANTIANXIETY | C |
| 0628 | ANTIPSYCHOTICS/ANTIMANICS | A |
| 0630 | ANTIDEPRESSANTS | A |
| 0631 | ANOREXIANTS/CNS STIMULANTS | D, C |
| 0632 | CNS, MISCELLANEOUS | C |
| 0633 | ALZHEIMER-TYPE DEMENTIA | C |
| 0634 | SLEEP AID PRODUCTS (OTC) | 0 |
| 0635 | ANTIEMETICS | A |
| 0700 | CONTRAST MEDIA/RADIOPHARMACEUTICALS | 0 |
| 0789 | DIAGNOSTICS, RADIOPAQUE & NONRADIOACTIVE | 0 |
| 0790 | DIAGNOSTICS—RADIOPHARMACEUTICALS | 0 |
| 0791 | THERAPEUTICS—RADIOPHARMACEUTICALS | 0 |
| 0792 | DIAGNOSTICS, MISCELLANEOUS | 0 |
| 0800 | GASTROINTESTINALS | A, C |
| 0874 | DISORDERS, ACID/PEPTIC | C |
| 0875 | ANTIDIARRHEALS | C |
| 0876 | LAXATIVES | C |
| 0877 | GASTROINTESTINAL, MISCELLANEOUS | C |
| 0878 | ANTISPASMODICS/ANTICHOLINERGICS | A |
| 0879 | ANTACIDS | C |
| 0900 | METABOLICS/NUTRIENTS | A, B, D |
| 0912 | HYPERLIPIDEMIA | B |
| 0913 | VITAMINS/MINERALS | A, 0 |
| 0914 | NUTRITION, ENTERAL/PARENTERAL | 0 |
| 0915 | REPL/REGS OF ELECTROLYTES/WATER BALANCE | A |
| 0916 | CALCIUM METABOLISM | B |
| 0917 | HEMATOPOIETIC GROWTH FACTORS | A |
| 1000 | HORMONES/HORMONAL MECHANISMS | B |
| 1032 | ADRENAL CORTICOSTEROIDS | A |
| 1033 | ANDROGENS/ANABOLIC STEROIDS | D |
| 1034 | ESTROGENS/PROGESTINS | B |
| 1035 | ANTERIOR PITUITARY/HYPOTHALMIC FUNCTION | B |
| 1036 | BLOOD GLUCOSE REGULATORS | A, B |
| 1037 | THYROID/ANTITHYROID | B |
| 1038 | ANTIDIURETICS | B |
| 1039 | RELAXANTS/STIMULANTS, UTERINE | A |
| 1040 | CONTRACEPTIVES | A |
| 1041 | INFERTILITY | 0 |
| 1042 | DRUGS USED IN DISORDERS OF GROWTH HORMONE SECRETION | D |
| 1100 | IMMUNOLOGICS | B |
| 1180 | VACCINES/ANTISERA | 0 |
| 1181 | IMMUNOMODULATORS | B |
| 1182 | ALLERGENIC EXTRACTS | 0 |
| 1183 | IMMUNE SERUMS | 0 |
| 1200 | SKIN/MUCOUS MEMBRANES | A, C, D |
| 1264 | ANTISEPTICS/DISINFECTANTS | 0 |
| 1265 | DERMATOLOGICS | D |
| 1266 | KERATOLYTICS | C |
| 1267 | ANTIPERSPIRANTS | C |
| 1268 | TOPICAL STEROIDS | A |
| 1269 | BURN/SUNBURN, SUNSCREEN/SUNTAN PRODUCTS | A, 0 |
| 1270 | ACNE PRODUCTS | D |
| 1271 | TOPICAL ANTI-INFECTIVES | A |
| 1272 | ANORECTAL PRODUCTS | C |
| 1273 | PERSONAL CARE PRODUCTS (VAGINAL) | 0 |
| 1274 | DERMATITIS/ANTIPURETICS | A |
| 1275 | TOPICAL ANALGESICS | C |
| 1300 | NEUROLOGICS | A, C |
| 1371 | EXTRAPYRAMIDAL MOVEMENT DISORDERS | C |
| 1372 | MYASTHENIA GRAVIS | C |
| 1373 | SKELETAL MUSCLE HYPERACTIVITY | C |
| 1374 | ANTICONVULSANTS | A |
| 1400 | ONCOLYTICS | B |
| 1479 | ANTINEOPLASTICS | B |
| 1480 | HORMONAL/BIOLOGICAL RESPONSE MODIFIERS | B |
| 1481 | ANTIMETABOLITES | B |
| 1482 | ANTIBIOTICS, ALKALOIDS, AND ENZYMES | B |
| 1483 | DNA DAMAGING DRUGS | B |
| 1500 | OPHTHALMICS | A, B, C |
| 1566 | GLAUCOMA | B |
| 1567 | CYCLOPLEGICS/MYDRIATICS | C |
| 1568 | OCULAR ANTI-INFECTIVE/ANTI-INFLAMMATORY | A |
| 1569 | OPHTHALMICS, MISCELLANEOUS | C |
| 1570 | OPHTHALMICS—DECONGESTANTS/ANTIALLERGY AGENTS | C |
| 1571 | CONTACT LENS PRODUCTS | 0 |
| 1600 | OTICS | A, C |
| 1670 | OTICS, TOPICAL | A |
| 1671 | VERTIGO/MOTION SICKNESS/VOMITING | C |
| 1700 | RELIEF OF PAIN | A, B, C |
| 1720 | ANALGESICS, GENERAL | C |
| 1721 | ANALGESICS—NARCOTIC | C |
| 1722 | ANALGESICS—NON-NARCOTIC | C |
| 1723 | ANTIMIGRAINE/OTHER HEADACHES | A |
| 1724 | ANTIARTHRITICS | B, C |
| 1725 | ANTIGOUT | B |
| 1726 | CENTRAL PAIN SYNDROMES | C |
| 1727 | NSAID | C |
| 1728 | ANTIPYRETICS | C |
| 1729 | MENSTRUAL PRODUCTS | C |
| 1800 | ANTIPARASITICS | A |
| 1860 | ANTIPROTOZOALS | A |
| 1862 | ANTHELMINTICS | A |
| 1863 | SCABICIDES/PEDICULICIDES | A |
| 1864 | ANTIMALARIALS | A |
| 1900 | RESPIRATORY TRACT | A, C |
| 1940 | ANTIASTHMATICS/BRONCODILATORS | A |
| 1941 | NASAL DECONGESTANTS | C |
| 1943 | ANTITUSSIVES/EXPECTORANTS/MUCOLYTICS | C |
| 1944 | ANTIHISTAMINES | C |
| 1945 | COLD REMEDIES | C |
| 1946 | LOZENGE PRODUCTS | C |
| 1947 | CORTICOSTEROIDS—INHALATION/NASAL | C |
| 2000 | UNCLASSIFIED/MISCELLANEOUS | 0 |
| 2087 | UNCLASSIFIED | D |
| 2095 | PHARMACEUTICAL AIDS | 0 |
| 2096 | SURGICAL AIDS | 0 |
| 2097 | DENTAL PREPARATIONS | 0 |
| 2098 | DENTRIFICE/DENTURE PRODUCTS | 0 |
| 2099 | MOUTH PAIN, COLD SORE, CANKER SORE PRODUCTS | A |
| 2100 | HOMEOPATHIC PRODUCTS | 0 |

In order to use Table 4 as said second list 536 in the above described method for assigning scripts to Groups, a suitable first list 534 of scripts assigned to FDA drug therapy codes must be provided.

The information for compiling said first list is found at: www.fda.gov/cder/ndc/index.htm (last viewed Apr., 12, 2004).

Said page is archived at: http://web.archive.org/web/20030202075941/http://www.fda.gov/cder/ndc/index.h tm (archived on Feb. 2, 2003) (Last viewed on Mar. 6, 2005)

Script identifiers suitable for the adjudication of pharmacy claims comprise the FDA NDC codes. The FDA NDC codes can be assigned to FDA drug therapy classes using the data found in tables LISTINGS.TXT and DRUGCLAS.TXT. LISTINGS.TXT provides FDA NDC codes assigned to the unique identifiers called LISTING_SEQ_NO. DRUGCLAS.TXT provides FDA drug class codes also assigned to LISTING_SEQ_NO. A list of FDA NDC codes assigned to FDA drug class numbers, therefore, can be produced using the LISTING_SEQ_NOs as a key.

An archived copy of LISTING.TXT can be found at: http://web.archive.org/web/20030202105905/www.fda.gov/cder/ndc/listings.txt

An archived copy of DRUGCLAS.TXT can be found at: http://web.archive.org/web/20030202103743www.fda.gov/cder/ndc/drugclas.txt

Both of said web pages were archived on Feb. 2, 2003 and last viewed on Mar. 6, 2005. They and the contents of any links therefrom are incorporated herein by reference.

New drugs that are added to the National Drug Code Directory can be automatically assigned to a default Group, such as Lifestyle, until such time as they can be more properly assigned, if necessary, to another Group. New drugs and their associated scripts may alternatively be assigned a default Group based on a Group assigned to the major class.

Certain private publishers produce proprietary databases which assign scripts with different NDC codes into their own hierarchical therapy classification schemes. These private publishers include First Data Bank of San Bruno Calif., Medi-Span of Indianapolis, Ind. and U.S. Pharmacopeia of Rockville, Md. These therapeutic databases may be used in a similar manner to the FDA therapeutic database to assign each script to a Group. The private databases provide more levels in their hierarchy than the FDA does thus reducing the incidence of scripts within a Class falling into multiple Groups. If scripts within a given Class fall into more than one Group, the next level in the therapy hierarchy can be examined to see if the Classes at that level fall into single Groups.

Provision can be made for the assignment of specific scripts with multiple indications to a single Group despite the fact that the multiple indications might fall into different Groups. For example, the proton pump inhibitor, Prilosec®, is often prescribed to treat acid reflux. Prilosec is made by AstraZeneca of London England. In most of the prescriptions written for Prilosec, the insured is merely suffering discomfort. Hence Prilosec might be put in the Workplace Group. A minority of the prescriptions for Prilosec, however, are prescribed for erosive acid reflux. This condition could likely lead to a covered medical expense, such as an emergency room visit or a hospital stay, within a year if left untreated. Hence Prilosec might also be put in the Acute Group.

One means of resolving this ambiguity in the assignment of a single Group to a given script is to review the actual volume of said scripts prescribed for the indications corresponding to different Groups. Said script could then be assigned to the Group with the most volume. This review can be accomplished by matching insurance claims for medical treatments received by individual insureds to pharmacy claims by the same insureds. The data analysis can be performed using known computer means. In the case of Prilosec, for example, a review of an insurance company's historical data showed that 80% of the prescriptions were for patients suffering from discomfort (Workplace Group). The remaining 20% were for patients suffering from erosive acid reflux (Acute Group). Hence all scripts of Prilosec could be assigned to the Group of Workplace. Additionally, provision could be made for individual patients suffering from erosive acid reflux to be granted and exception and have benefits for Prilosec set at the Acute level.

An alternative means of resolving an ambiguity in the Group that the scripts of a given Class might belong to is to review the effect of Group assignment on subsequent covered medical costs. In the case of Prilosec, for example, assignment of Prilosec to the Workplace Group with its corresponding relatively low allowance might result in more emergency room visits by the minority of insureds who were prescribed the drug for erosive reflux but did not take it due to its high out-of-pocket expense or the trouble of getting an exception. The high covered expense of those few additional emergency room visits might justify putting Prilosec in the Acute Group, even though most of those taking it did so merely to relieve discomfort.

Provision can also be made for alternative assignment of certain scripts to different Groups to account for societal norms or legal requirements of a particular set of insureds. In the United States, for example, birth control pills are categorized as Acute since in many States laws require that health plans provide benefits for the hospitalization expense of pregnancy. Birth control pills reduce the probability of an insured person incurring a subsequent medical cost for pregnancy within a year. Hence birth control pills are categorized as Acute.

In other jurisdictions, however, the medical costs of pregnancy might not be covered by a health insurance plan. Hence birth control pills might be categorized as Workplace or Lifestyle in those jurisdictions.

Provision can also be made for alternative assignment of certain scripts to different Groups to account for changes in societal norms or legal requirements of a set of insureds. At present, for example, obesity drugs are categorized as Lifestyle in the United States. It is conceivable that future medical research would indicate that obesity drugs should be categorized as Chronic if it can be shown that taking these drugs results in lower long-term health care costs.

Provision can also be made to categorize scripts according to the particular diagnosis for which they were prescribed. A physician, for example, could indicate a diagnosis on a script. At the time of adjudication of a claim for said script, the particular diagnosis for the script would be matched to a corresponding Group. The benefits would be calculated according to said Group.

### Claims Adjudication

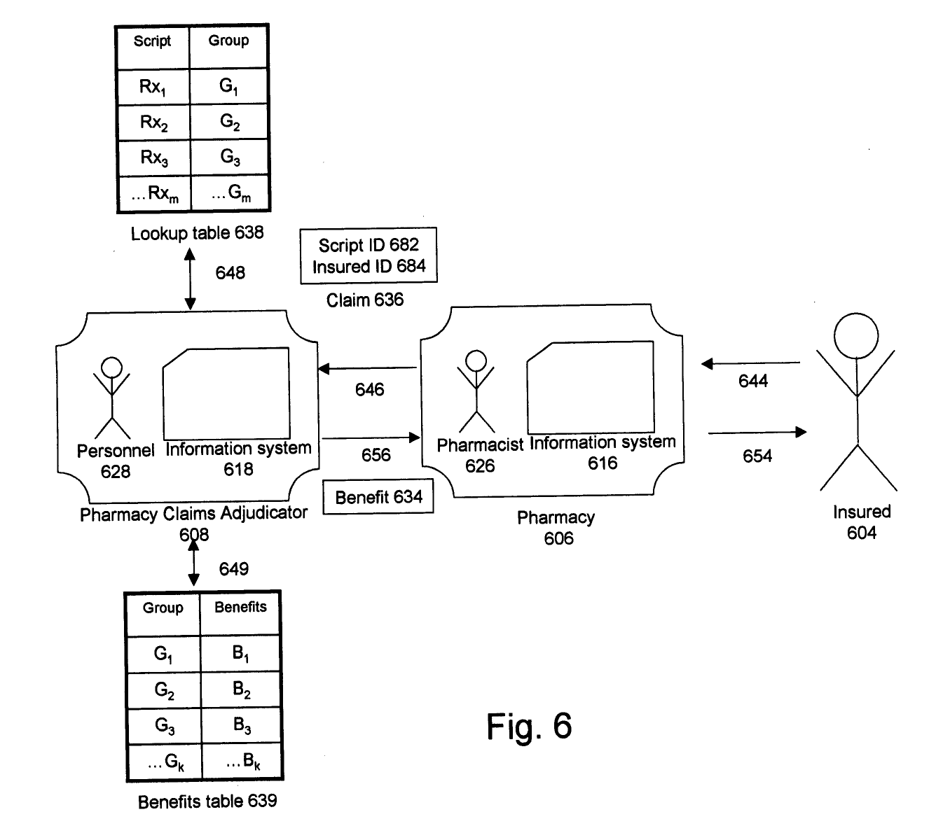

FIG. 6 illustrates a method of adjudicating pharmacy claims according to the present invention. A pharmacy claims adjudicator 608 may comprise personnel 628 and an information system 618.

A method of adjudicating pharmacy claims comprises:
a) said pharmacy claims adjudicator 608 receiving 646 a pharmacy claim from an insured 604, said pharmacy claim 636 comprising an identifier 682 of a given script and an identifier 684 of said insured;
b) accessing 648 a lookup table 638, said lookup table comprising a list of identifiers of scripts and corresponding assignments of said scripts to Groups, said assignments being such that:
   i. said Groups comprise a first Major Group;
   ii. said scripts assigned to said first Major Group comprise scripts for drugs that are prescribed for medical conditions that would otherwise likely require subsequent medical costs within one year of being prescribed if said drugs were not taken by said insured when prescribed; and
   iii. at least two or more of said scripts assigned to said first Major Group are also assigned to two or more different therapy classification codes;
c) accessing 649 a benefits table 639, said benefits table comprising benefit levels for a given Group appropriate for said insured.
d) determining the Group that is assigned to said given script;
e) determining a benefit corresponding to said Group;
f) approving 656 the payment of said benefit 634 to said insured; wherein at least one of said steps is carried out at least in part performed by an information system.

The method may additionally comprise a pharmacy 606 acting as an intermediary 644, 654 between said given insured and said pharmacy claims adjudicator. The pharmacy may comprise a pharmacist 626 and information system 616.

### Product Acceptance by Insureds

A number of additional features can be provided with the instant invention to improve its acceptance by a set of prospective insureds. These features may also be incorporated in other pharmacy benefits programs apart from those described herein.

### Benefits Calculator

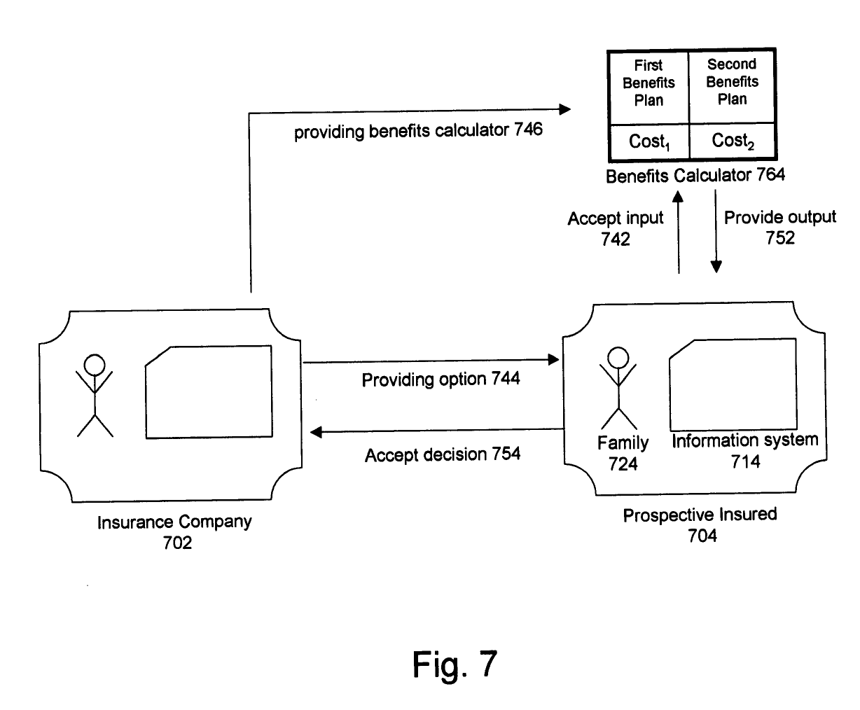

FIG. 7 illustrates how the acceptance of a pharmacy benefits plan according to the present invention can be surprisingly improved by providing an online pharmacy benefits calculator when offering the inventive pharmacy benefits plan to a set of prospective insureds. The benefits calculator allows said prospective insureds to compare their out-of-pocket costs under their current pharmacy benefits plan with their out-of-pocket costs for the inventive pharmacy benefits plan.

A method for an insurance company 702 or other entity to offer a first pharmacy benefits plan to a prospective insured 704 comprises:
a) providing 744 an option to said prospective insured to select between said first pharmacy benefits plan and at least one second pharmacy benefits plan;
b) providing 746 a benefits Calculator 764 to said prospective insured, said benefits Calculator adapted to:
   i. accept 742 input comprising the expected pharmacy needs of said prospective insured; and
   ii. provide 752 output comprising the expected out-of-pocket costs (Cost₁, Cost₂) of said prospective insured under said first pharmacy benefits plan and said at least one second pharmacy benefits plan;
c) accepting 754 a decision from said prospective insured as to which of said first or said at least one second pharmacy benefits plan said prospective insured wishes to enroll in,
   wherein at least one of said steps at carried out using an information system.

Said prospective insured may comprise one or more people 724, such as the members of a family. Said prospective insured may also comprise an information system 714, such as a personal computer connected to the Internet.

When using the pharmacy benefits calculator, a prospective insured would input their anticipated pharmacy needs for an upcoming plan year and compare their out-of-pocket costs with different benefits options. The different benefits options may include different reimbursement levels for different Groups, different limits to an insured's total out-of-pocket pharmacy costs for a given year, and different suggested lower cost alternatives to a given choice of drugs and dosages.

The benefits calculator can be enabled with a spreadsheet. The benefits calculator may read in historical data for a given prospective insured in order to save said prospective insured the trouble of finding and inputting their own data. The historical data would reside in a database that the benefits calculator can access. Access to said database may be protected by known security means, such as a user ID and password. Transmission of data may be by encryption such that unauthorized personnel cannot read said data.

It has been found that offering the inventive pharmacy benefits plan for the first time as an alternative to an existing pharmacy benefits plan using said benefits calculator resulted in a surprisingly high acceptance rate of the inventive plan. First year acceptance among the employees of a major corporation was 18%, as opposed to normal first year acceptance rates of new plans of 5%.

### Out-of-Pocket Limits

Another feature that contributes to the acceptance of this invention by prospective insureds is provision for the prospective insureds to select their own limits for out-of-pocket expenses.

The limits may be an upper limit per prescription. $75 is a suitable default for the out-of-pocket limit for a single prescription where the average price of prescriptions is $40. A suitable range for allowable out-of-pocket limits is $75 to $200, or 190% to 500% of the average cost of all scripts.

A prospective insured may select a higher or lower limit than the default, depending upon their personal needs. A prospective insured selecting a lower limit would pay a higher share of the premium for their employer sponsored insurance coverage and vice versa.

There may also be limits for total annual out-of-pocket expenses. A suitable range for the limit of total annual out-of-pocket pharmacy expenses for each prospective insured is in the range of $1,500 to $3,000 for an average annual pharmacy cost per prospective insured of about $500. This corresponds to a range of suitable upper limits for annual out-of-pocket costs of 300% to 600% of average annual pharmacy cost per prospective insured.

The upper limit can also be selected based on the percentage of prospective insureds that might exceed the upper limit. A range of 1 to 5% of the prospective insureds exceeding the upper limit is suitable. Provision may also be made for prospective insureds to select their own out of pocket limits with corresponding adjustments in their share of the insurance premiums.

The relationship between premiums and out-of-pocket limits can be calculated using known actuarial techniques and historical pharmacy benefits data.

The portion of the premiums paid by an employer can be fixed such that the portion of the premium paid by an employee would vary according to the out-of-pocket limits selected.

The provision of out-of-pocket limits in combination with the present invention for providing a given allowance for a given script based on the Group that a script belongs to has resulted in a surprising increase in product acceptance by a set of prospective insureds. The enrollment rate in the inventive pharmacy benefits plan increased by 400% in the year that out-of-pocket limits were introduced.

### Accumulating Credit for Unused Allowances

Another feature that contributes to the acceptance of this invention by insureds is provision for the insureds to accumulate credit for the unused portion of a given allowance provided for a given script. It has been observed, for example, that the median unused benefit per claim is $11 when an allowance of $30 is provide for scripts with an average cost of $40 per script. The reason why there is commonly an unused benefit is that most scripts cost less than $30 even though the average cost per script is $40. The reason is that a small number of very expensive scripts pull the average up.

The inventive pharmacy benefits plan, or any pharmacy benefits plan based on allowances, will be more attractive to prospective insureds if a low cost, convenient method is provided to accumulate the unused portions of allowances provided for low cost scripts to help offset the cost of the occasional high cost scripts. Said unused portions of said allowances might also be used to offset the out-of-pocket costs of other covered medical expenses or medical expenses that are not covered but nonetheless have favored tax treatment such as that provided under the US tax codes for residents of the United States.

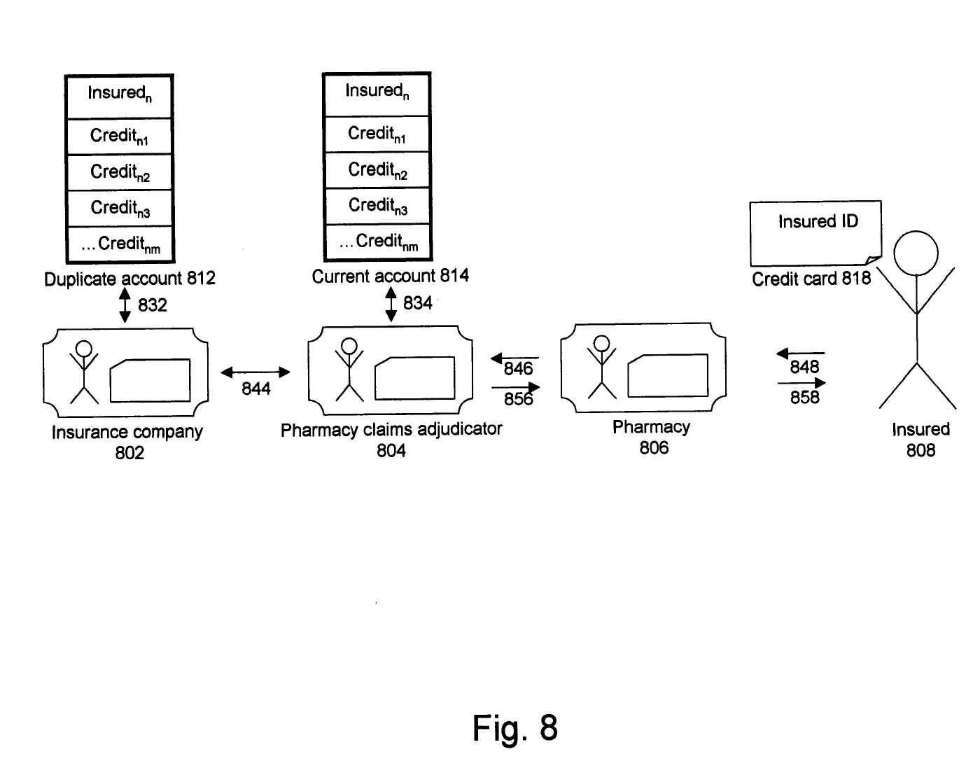

A suitable method for accumulating and redeeming credits for unused allowance benefits is illustrated in FIG. 8.

A method of providing benefits to an insured 808 enrolled in a pharmacy benefits plan, said pharmacy benefits plan providing an allowance for a covered script, comprises:
a) providing a means to accumulate a credit for a claim 848 made by said insured against said pharmacy benefits plan, said claim being for the purchase of said covered script, said credit being equal to the positive difference between said allowance for said covered script and the cost of said covered script; and
b) providing a means for said insured to redeem at least a portion of said accumulated credit for the purchase of an allowed expense,
   wherein at least one of said steps is at least in part carried out using an information system.

When an insured 808 purchases a covered script, said insured provides 848 a credit card 818 to a pharmacy 806. The pharmacy transmits 846 the Insured ID found on said credit card and ID information related to the purchase of said script to a pharmacy claims adjudicator 804. The pharmacy claims adjudicator then determines the appropriate allowance for said script and whether or not there is an excess that should be accumulated 834 in an account 814 associated with said insured (insuredₙ). If there is an excess, then said account is so credited (Creditₙₘ). If there is a deficit, then the pharmacy claims adjudicator can check to see if there is an accumulated credit in said insured's account that can be used to offset said deficit. If so, then the deficit is reduced, if not eliminated, and the insured's account is adjusted accordingly. The pharmacy claims adjudicator then transmits 856 the appropriate information to the pharmacy. The pharmacy then, in turn, communicates 858 the information to the insured.

Periodically, the pharmacy claims adjudicator will update 844 the insurance company 802 of changes in an insured's current account. The insurance company will, in turn update 832 their own duplicate account files 812 of the insured. The insurance company will also periodically update the pharmacy claims adjudicator of changes on their end, such as the enrollment of a new insured or the dropping of an existing insured.

The credit card used by the insured may also be a smart card that has provision to store an account balance directly on it. The credit card may also function as a normal credit card, such as MasterCard®, issued by a credit card company.

The combined features of the present invention, including the provision of a means for accumulating credit has resulted in a surprising reduction in the frequency of exceptions requested by insureds. A conventional plan based on copayments related to whether or not a given script if for a brand name or generic drug had been experiencing an exception rate of about 10% of all claims. This required the provision of about 50 personnel and associated information systems to accommodate the high number of doctor requests for approvals to exceptions to the standard copay benefits coverage. When the same population switched to the inventive plan comprising allowances paid for scripts according to the Groups the scripts were assigned to and the provision of a means to accumulate credit for the unused portion of a given script, the exception rate dropped to below 2% for all claims.

### Funding Level

The present invention is suitable for employers offering health care benefits to their employees. Employers pay a certain portion of the premiums and employees pay the balance of the premiums. The employees may pay their premiums through a payroll deduction. A suitable range of employer share of premiums is in the range of 50% to 100% of the total premium. A preferred fraction of the total premiums paid by the employer may be 80%.

The invention may also be suitable for medical plans offered directly to consumers. In that case the consumer pays 100% of the premium.

The invention may also be suitable for medical plans offered by governments, such as universal health care plans.

### EXAMPLE

A pharmacy benefits plan according to the present invention was offered as an alternative to a conventional pharmacy benefits plan to the United States employees of a publicly traded corporation. The benefits for the inventive program were calculated according to the Groups that specific scripts were assigned to. Specific drugs were assigned to Groups according to Table 5. Said benefits comprised allowances paid for drugs, said allowances being based on the Group a given drug was assigned to.

Based on prior experience with offering new pharmacy benefits plans, it was anticipated that 5% or less of said employees would select the inventive pharmacy benefits plan in the first year it was offered. The low acceptance rate of new plans is believed to be primarily due to the unfamiliarity of a given set of employees with a given new plan and not necessarily due to the desirability of a new plan. Hence when the inventive plan was first offered to said employees, a Pharmacy Benefits Calculator was additionally provided via a Web interface so that said employees could more accurately project their personal out-of-pocket costs under the inventive plan and under their conventional plan. Surprisingly, 18% of said employees selected the inventive plan in the first year it was offered.

It was discovered that employees wished that they could accumulate the unused portions of their allowances so that said unused portions could be applied to other qualified out-of-pocket medical expenses. It was also discovered that employees wanted more protection against the possibility that they might have unusually high pharmacy costs. Hence in the second year that the inventive pharmacy benefits plan was offered, provision was made to allow employees to accumulate unused portions of their allowances and apply said unused portions to other qualified out-of-pocket medical expenses. Said provision comprised providing said employees with a special purpose credit card.

Said inventive pharmacy benefits program was also modified to provide a limit of $1,500 for an employee's out-of-pocket pharmacy expenses in a given year and a limit of $75 of an employee's out-of-pocket pharmacy expenses for a given script. The Pharmacy Benefits Calculator was modified to incorporate these changes. In the second year that the inventive pharmacy benefits plan was offered with the above improvements, the acceptance rate jumped to 45%.

A number of surprising benefits were observed with the widespread adoption of the inventive plan. The overall growth in pharmacy costs of employees enrolled in the inventive pharmacy benefits plan was significantly less than the national average growth in pharmacy costs. The exception rate dropped from 10% for employees enrolled in a conventional pharmacy benefits plan to less than 1% for employees enrolled in the inventive plan. There was a significant reduction in the time and effort required to negotiate discounts from drug manufacturers since the need for discounts to control the growth in pharmacy costs was greatly reduced.

### CONCLUSION

One of skill in the art will recognize that insurance is a regulated industry. One practicing the methods described and claimed herein will want to maintain compliance with all applicable local, state and federal regulations, to ensure that the insurance policy is properly presented to the insured, premiums are properly approved, underwriting properly occurs, all necessary regulatory approvals are in place, etc.

While particular embodiments of the present invention have been illustrated and described, it would be obvious to those skilled in the art that various other changes and modifications can be made without departing from the spirit and scope of the invention. Any of the aspects of the invention of the present invention found to offer advantages over the state of the art may be used separately or in any suitable combination to achieve some or all of the benefits of the invention disclosed herein.

LENGTHY TABLES FILED ON CD
The patent application contains a lengthy table section. A copy of the table is available in electronic form from the USPTO web site (http://seqdata.uspto.gov/?pageRequest=docDetail&DocID=US20070043589A1). An electronic copy of the table will also be available from the USPTO upon request and payment of the fee set forth in 37 CFR 1.19(b)(3).

We claim:

1. A method of assigning pharmacy scripts into Groups, said Groups being useful to a pharmacy claims adjudicator for determining the benefits payable for one or more pharmacy claims made by an insured person, said method comprising:
a) receiving a first list of said scripts, said list comprising therapy classification codes assigned to at least a portion of said scripts;
b) receiving a second list of said therapy classification codes, said list comprising Groups assigned to at least a portion of said therapy classification codes wherein;
i. said Groups comprise a first Major Group;
ii. said therapy classification codes assigned to said first Major Group correspond to scripts for drugs that are prescribed for medical conditions that would otherwise likely require subsequent medical costs within one year of being prescribed if said drugs were not taken by said insured when prescribed; and
iii. said first Major Group is assigned to at least two or more different therapy classification codes in said second list; and
c) providing a third list to said pharmacy claims adjudicator, said third list being formed by assigning said Groups of said second list to said scripts of said first list using said therapy classification codes as a key;
wherein at least one of said steps is carried out at least in part by an information system.

2. The method of claim 1 wherein said therapy classification codes are hierarchical therapy classification codes.

3. The method of claim 2 wherein:
a) said hierarchical therapy classification codes comprise one or more major classes; and
b) said assignment of said Groups to said therapy classification codes is based at least in part on the major class of at least a portion of said therapy classification codes.

4. The method of claim 2 wherein
a) said hierarchical therapy classification codes comprise one or more minor classes; and
b) said assignment of said Groups to said therapy classification codes is based at least in part on the minor classes of at least a portion of said therapy classification codes.

5. The method of claim 1 wherein said Groups comprise a second Major Group wherein:
a) said therapy classification codes assigned to said second Major Group correspond to scripts that are prescribed for medical conditions that would not normally require subsequent health care cost by said insured if said insured did not take said scripts with prescribed.

6. The method of claim 5 wherein the scripts assigned to said first Major Group comprise at least one generic script and at least one brand name script.

7. The method of claim 5 wherein said first Major Group is assigned to at least three or more of the therapy classification codes corresponding to:
a) Acute electrolyte drugs
b) Allergic reaction steroids
c) Anaphylaxis drugs
d) Antibiotics
e) Anti-convulsants
f) Anti-depressants
g) Antidote drugs
h) Anti-emetics
i) Anti-Microbial-PPI combination drugs
j) Anti-Psychotics
k) Asthma drugs
l) Blood Clotting Modifiers
m) Contraceptives
n) Diabetic supplies/insulin
o) Hematologic Growth Factors
p) HIV drugs
q) Migraine drugs
r) Ophthalmic—Steroids and Anti-infectives
s) Otic—anti-infectives
t) Rx Vitamins/prenatal
u) Topical—anti-infectives

8. The method of claim 5 wherein said second Major Group is assigned to at least three or more of the therapy classification codes corresponding to:
a) Alzheimer's drugs
b) Amphetamines
c) Analgesics
d) Anti-anxiety drugs
e) Anti-diarrheal drugs
f) Cough/Cold/Allergy
g) Influenza
h) Laxatives
i) Muscle relaxation drugs
j) Narcotics
k) Non-sedating antihistamines
l) Non-Steroid Anti-inflammatory drug
m) Ophthalmic-13 Decongestants
n) Parkinson's disease drugs
o) Prostate Hypertrophy
p) Proton Pump drugs/H-2 drugs used to reduce stomach acid.
q) Topical Anesthetics

9. A method of adjudicating pharmacy claims, said method comprising:
a) receiving a pharmacy claim from an insured, said pharmacy claim comprising an identifier of a given script and an identifier of said insured;
b) accessing a lookup table, said lookup table comprising a list of identifiers of scripts and corresponding assignments of said scripts to Groups, said assignments being such that:
i. said Groups comprise a first Major Group;
ii. said scripts assigned to said first Major Group comprise scripts for drugs that are prescribed for medical conditions that would otherwise likely require subsequent medical costs within one year of being prescribed if said drugs were not taken by said insured when prescribed; and
iii. at least two or more of said scripts assigned to said first Major Group are also assigned to two or more different therapy classification codes;
c) accessing a benefits table, said benefits table comprising benefits levels for a given Group appropriate to said insured;
d) determining the Group that is assigned to said given script;
e) determining a benefit corresponding to said Group;
f) approving the payment of said benefit to said insured;
wherein at least one of said steps is carried out at least in part using an information system.

10. The method of claim 9 wherein said Groups comprise a second Major Group and wherein the scripts assigned to said second Major Group comprise scripts that are prescribed for medical conditions that would otherwise likely require subsequent medical costs within one year to five years of being prescribed.

11. The method of claim 10 wherein the benefit corresponding to said first Major Group comprises an allowance, said allowance being sufficient enough to cover the cost of most therapeutic alternatives to a given script, but not the most expensive effective alternative.

12. The method of claim 11 wherein said allowance is 25% or more of the average cost of all scripts assigned to said first Major Group.

13. The method of claim 12 wherein said allowance is 150% or less of the average cost of all scripts assigned to said first Major Group.

14. A method of offering a first pharmacy benefits plan to a prospective insured, said method comprising:
a) providing an option to said prospective insured to select between said first pharmacy benefits plan and at least one second pharmacy benefits plan;
b) providing a benefits Calculator to said prospective insured, said benefits Calculator adapted to:
i. accept input comprising the expected pharmacy needs of said prospective insured; and
ii. provide output comprising the expected out-of-pocket costs of said prospective insured under said first pharmacy benefits plan and said at least one second pharmacy benefits plan;
c) accepting a decision from said prospective insured as to which of said first or said at least one second pharmacy benefits plan said prospective insured wishes to enroll in,
wherein at least one of said steps at carried out using an information system.

15. The method of claim 14 wherein said first pharmacy benefits plan has scripts assigned to Groups wherein;
a) said Groups comprise a first Major Group such that the scripts assigned to said first Major Group comprise scripts that are prescribed for medical conditions that would otherwise likely require subsequent medical costs within one year of being prescribed; and
b) at least two or more of said scripts assigned to said first Major Group are also assigned to two or more different therapy classification codes.

16. The method of claim 14 wherein said benefits Calculator is further adapted to read in historical data regarding said prospective insured's prior pharmacy needs, said data residing on a database accessible to said benefits Calculator.

17. The method of claim 14 wherein said benefits Calculator is adapted to provide different suggested lower cost alternatives to a said prospective insured's expected pharmacy needs.

18. The method of claim 14 wherein said benefits Calculator comprises a spreadsheet.

19. A method of providing benefits to an insured enrolled in a pharmacy benefits plan, said pharmacy benefits plan providing an allowance for a covered script, said method comprising:
a) providing a means to accumulate a credit for a claim made by said insured against said pharmacy benefits plan, said claim being for the purchase of said covered script, said credit being equal to the positive difference between said allowance for said covered script and the cost of said covered script; and
b) providing a means for said insured to redeem at least a portion of said accumulated credit for the purchase of an allowed expense,
wherein at least one of said steps is at least in part carried out using an information system.

20. The method of claim 19 wherein said pharmacy benefits plan has scripts assigned to Groups wherein:
a) said Groups comprise a first Major Group such that the scripts assigned to said first Major Group comprise scripts for drugs that are prescribed for medical conditions that would otherwise likely require subsequent medical costs within one year of being prescribed to said insured if said insured did not take said drugs; and
b) at least two or more of said scripts assigned to said first Major Group are also assigned to two or more different therapy classification codes.

21. The method of claim 19 wherein said allowed expenses comprise:
a) the cost of a script covered by said pharmacy benefits plan;
b) medical expenses covered by a health insurance policy; or
c) medical expenses that meet the US Internal Revenue Service (IRS) requirements for favored tax treatment.

22. The method of claim 19 wherein the accumulated credits persist for more than one year.

23. The method of claim 19 wherein said step of providing a means for said insured to redeem at least a portion of said accumulated credit comprises providing a credit card to said insured, said credit card being acceptable by one or more vendors for payment of an allowed expense using said accumulated credit.
